(19) United States
(12) Patent Application Publication
(10) Pub. No.: US 2007/0094052 A1
(43) Pub. Date: Apr. 26, 2007

(54) Medical imaging and report management

(76) Inventor: Robert Blas, Snellville, GA (US)

Correspondence Address: NELSON MULLINS RILEY & SCARBOROUGH, LLP, 1320 MAIN STREET, 17TH FLOOR, COLUMBIA, SC, 29201, US

(21) Appl. No.: 11/433,956

(22) Filed: May 15, 2006

Related U.S. Application Data
(60) Provisional application No. 60/680,680, filed on May 13, 2005.

(51) Int. Cl. G06Q 10/00; A61B 5/00; G06F 7/00; H04N 7/173; G06F 19/00; G06F 17/30; G06Q 50/00

(52) U.S. Cl. 705/3; 725/86; 707/1; 705/2

(57) ABSTRACT

This invention provides methods of capturing, transporting, and reporting video patient studies using electronic networks. A medical image reporting system may include a data harvester that receives digital video clips for review by a radiologist, cardiologist, or other specialist, an application that provides a convenient user interface for the user to create diagnosis statements and Digital Imaging and Communications (“DICOM”) compatible data, an overview engine that allows review of the report statements and correction of entries, and a data packager that packages and transports the preliminary report, video clips, and/or patient information electronically to a data center, which then offers electronic access to authorized physicians and specialists located remotely. Some embodiments involve the use a foot pedal or other foot-operated component to trigger or otherwise control the capture of video images.

### RELATED APPLICATIONS

This document claims the benefit of U.S. Provisional Application Ser. No. 60/680,680, entitled “Medical Imaging and Report Management” and filed May 13, 2005, the entire contents of which are incorporated by this reference.

### FIELD OF THE INVENTION

The invention generally relates to video-recorded patient studies and medical reporting. More specifically, the invention relates to systems and methods of capturing, transporting, and reporting medical data, images, and videos related to patient studies.

### BACKGROUND

Ultrasound imaging, also called ultrasound scanning or sonography, is a method of obtaining images from inside the human body through the use of high frequency sound waves. Reflected sound wave echoes are recorded and displayed as a real-time visual image. Ultrasound is a useful way of examining many of the body's internal organs, including the heart, liver, gallbladder, spleen, pancreas, kidneys, and bladder. Because ultrasound images are captured in real-time, they can show movement of internal tissues and organs, and enable physicians to see movement such as blood flow and heart valve functions. This can help to diagnose a variety of heart conditions and to assess damage after a heart attack or other illness. Ultrasound procedures are typically performed by a technologist or radiologist. However, the procedures may also be performed by a cardiologist, or other specialist. The operator presses a transducer firmly against the patient's skin and sweeps it back and forth to image the area of interest. The image is typically displayed on a TV monitor and may also be recorded. In some cases, the technologist or radiologist is able to review and assess the ultrasound images in real time as they are acquired, and the patient can be released immediately. In other cases, when the examination is complete, the patient may be asked to dress and wait while the recorded ultrasound images are reviewed by the technologist or radiologist. In still other cases, the image must be reviewed by remotely located persons and the patient is dismissed to be contacted at a later time with the results.

In the case of cardiac ultrasound imaging, the cardiac ultrasound video is traditionally recorded on one or more video cassette tapes. The technologist performing the study typically attaches to the VCR tape a handwritten preliminary report describing his or her findings based on a review of the images. The tape and report are then sent to a cardiologist for reading. In some cases, the cardiologist may be in-house or local. In other cases, the tape and report are physically transported long distances to a remotely located cardiologist for reading. In still other cases, the cardiologist may travel to read echoes at multiple sites, wasting valuable time and resources.

Recently, medical professionals have begun to take advantage of the new formats and techniques available for capturing and transmitting information electronically, such as digital imaging and network communications. However, there is a continuing need for improved medical image reporting systems and methods that facilitate and manage electronic medical image creation, use, and reporting. It is also advantageous to improve medical imaging and transcription technologies in order to take advantage of very remote resources, such as lower-cost labor and medical specialists available in foreign countries and other places to which physical shipment of information is impractical.

### SUMMARY

Certain embodiments of the present invention utilize digital technology and the Internet for capturing images, transporting information, and reporting patient studies in a fast and efficient manner. The invention facilitates the production of a preliminary report and a final diagnosis report and extends the geographic boundaries of medical personnel involved in the capturing, evaluation, and reporting of medical images. A study, preliminary report, and/or final diagnostic report may be produced and available to a referring physician within minutes or hours as compared to the multi-day delays of traditional methods and processes. The images and reports may also be signed by the respective technologist, doctor, or specialist.

In certain embodiments, a medical image reporting system for video-recorded patient studies includes a data harvester that receives digital video clips, a preliminary report generator, a data packager, and a data center. The preliminary report generator may include a user interface for creating a preliminary report of diagnosis statements and/or a review engine that allows review and correction of the statements. The data packager may package and transport the preliminary report and video clips and may act as a short-term image repository and streaming media server to provide electronic access to the preliminary report and video clips through various networks. The data center receives the preliminary report and offers electronic access to the preliminary report by authorized users. The system may also connect to one or more medical information systems to retrieve general patient information and may include a final report generator for providing a final report to an interested party.

Certain embodiments of the invention include a method of managing a video-recorded patient study that involves receiving digital video clips, generating a preliminary report, reviewing diagnosis statements of the preliminary report, submitting the preliminary report and video clips to a data packager, packaging and transporting the preliminary report and video clips from the data packager to a data center, offering electronic access from the data center and through various networks to the preliminary report by authorized users, and providing a final report to an interested party.

According to certain embodiments, the invention also involves a method of managing a video-recorded ultrasound patient study that involves acquiring an ultrasound video, evaluating the ultrasound video to create a preliminary report, reviewing the preliminary report, and sending the report to an archiving system. Evaluating the ultrasound video may involve presenting the ultrasound video to a first user through a user interface, accepting one or more diagnosis statements, and/or storing the diagnosis statements as a preliminary report. Reviewing the preliminary report may involve presenting the ultrasound video and diagnosis statements to a second user through a user interface, allowing changes to the diagnosis statements by the second user, and/or storing a revised preliminary report. Sending the report to an archiving system may involve, at a data packager, packaging and encrypting the ultrasound video and revised preliminary report from a data packager to an archiving and image management (AIM) data center. At the AIM data center, the ultrasound video and revised preliminary report may be decrypted and stored in one or more databases and provided to authorized users over various networks. The ultrasound video and revised preliminary report may also be replicated at additional AIM data centers. The method may further involve allowing authorized users to modify the diagnosis statements to generate a final report by providing the ultrasound video and revised preliminary report to authorized users, allowing changes to the diagnosis statements, storing a final report at the AIM data center, and replicating the final report at additional AIM data centers.

Certain embodiments involve a method of providing AIM from a data center that involves storing videos and reports at one or more database servers, providing the stored videos and reports to authorized users through various networks. For a given user request, a video and report are provided by authenticating the user, determining the connection speed of the user, encoding the video and report based on connection speed, and/or sending the encoded video and report to the user.

Another aspect of the invention may involve a device for use in capturing video that includes two or more foot-operated components that each control different forms of video capture, e.g., video capture for a predefined amount of time, still image capture, and start/stop video capture.

### BRIEF DESCRIPTION OF THE DRAWINGS

These and other features, aspects, and advantages of the present invention are better understood when the following Detailed Description is read with reference to the accompanying drawings, wherein:

FIG. 1 illustrates a medical imaging and report management system according to certain embodiments of the present invention;

FIG. 2 illustrates the interaction between the ultrasound and data harvester in certain embodiments of the present invention;

FIG. 3 illustrates the interaction between the data harvester, foot pedal, and a switch in certain embodiments of the present invention;

FIG. 4 illustrates a data packager and a switch of certain embodiments of the present invention;

FIG. 5 illustrates an exemplary network that includes an AIM component in accordance with certain embodiments of the present invention; and

FIG. 6 illustrates an exemplary user interface display.

### DETAILED DESCRIPTION OF SPECIFIC EMBODIMENTS

I. Introduction

The present invention relates to improvements for video-recorded patient studies and reporting. It involves the use of various electronic components and methods to more quickly and efficiently capture, transport, and report patient studies (like the cardiac ultrasound video analysis) across a wide range of electronic networks, including the Internet. Certain embodiments of the present invention include a medical image reporting system including a data harvester that receives digital video clips for review by a radiologist, cardiologist, or other specialist, an application that provides a convenient user interface for the radiologist, cardiologist, or other specialist to create diagnosis statements and Digital Imaging and Communications (“DICOM”) compatible data, an overview engine that allows review of the report statements and correction of entries, and a data packager that packages and transports the preliminary report, video clips, and/or patient information electronically to a data center, which then offers electronic access to authorized physicians and specialists located remotely.

Other embodiments of the invention involve the use of a foot pedal to trigger or otherwise control the capture of video images by the sonographer. The foot pedal has buttons that allow various alternative forms of video capture. For example, a default capture button could record for a predefined amount of time (e.g. 3-4 seconds), a still capture button could capture a still image of the current study, and a start/stop capture button could enable the technologist to control the start and stop of the recording. The foot pedal allows the physician to control the video without occupying his or her hands.

II. System Overview

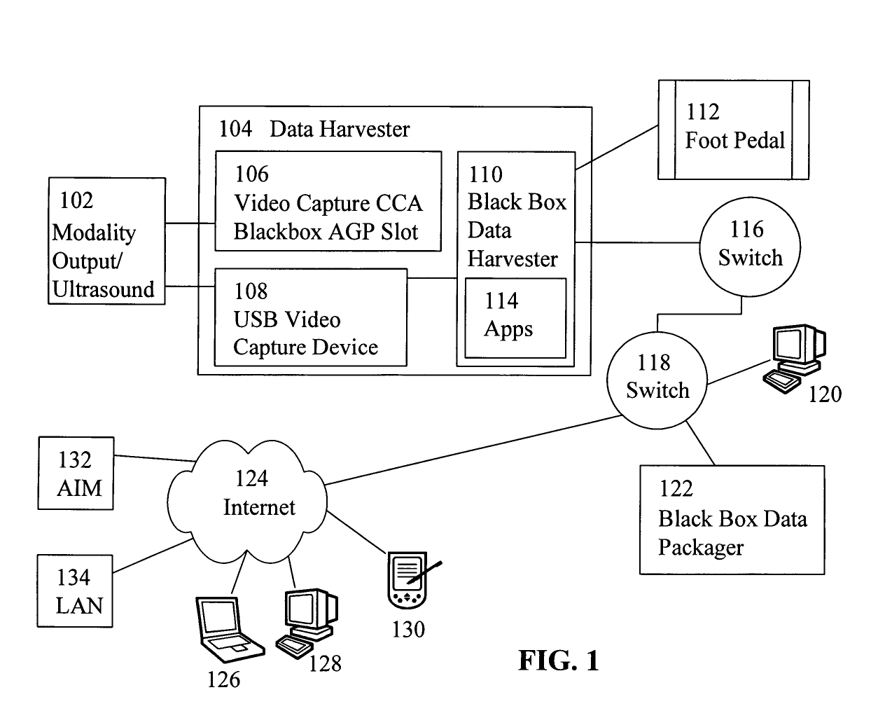

FIG. 1 illustrates a medical imaging and report management system according to certain embodiments of the present invention. A technologist, radiologist, cardiologist, or other specialist may use the system to create video clips, such as ultrasound video clips, to aid in preparation of a preliminary report. The ultrasound 102 output video clips are sent to data harvester 104. In some cases, DICOM data will also be sent to the data harvester 104. In this embodiment, data harvester 104 includes a video capture CCA blackbox AGP slot 106, a USB video capture device 108, and a blackbox data harvester 110. The data harvester 104 is connected to a foot pedal 112, which allows the technologist, radiologist, cardiologist, or other specialist to control video capture. The blackbox data harvester 110 may contain one or more applications 114, such as a report generator application. Captured video data or clips are sent through a first switch 116 and a second switch 118. At switch 118, the captured video data is available to one or more local computers 120, to black box data packager 122. These components allow the radiologist, technologist, cardiologist, or other specialist to prepare a preliminary report. The preliminary report along with the captured video data may be made available on the Internet 124 and to one or more local computers 118. The report and data maybe sent to a data center that acts as a data repository.

Remote computing devices, such as a laptop 126, desktop 128, or pda 130, may be used to access the preliminary report and captured video data over the Internet 124. The remote computing devices may be connected to the Internet in any suitable manner. In addition, the preliminary report and captured video data may be sent to a computing device offering an Archive and Image Management (AIM) 132 system. Such an AIM system and components may act as a data center providing a repository of data that is accessible by authorized physicians electronically, e.g., via portals. The network may also include various local area network (LAN) 134 and similar components through which physicians, surgeons, and other interested parties access the Internet and the AIM system. Accordingly, remotely located surgeons, doctors, and other interested parties may access the preliminary report and captured video data at a laptop 126, desktop 128, or pda 130, and/or may utilize a specific AIM application to access, alter, report, or otherwise use the relevant information. For example, a surgeon may access and use a preliminary report to prepare a final report and then make that final report available electronically to a patient.

The system of this embodiment may also incorporate connection to other medical information systems. For example, the system may connect to and retrieve general patient information that may be included in the preliminary or final report. Embodiments of the invention also provide various user interface, image capture, and report management methods and systems that facilitate and improve users' abilities to efficiently create, use, track, manage, and store patient information. Some embodiments include providing the final report electronically to the patient, a local physician, or other interested party such as an insurance or government agency.

In general, the logical and device components of the invention can exist as computer and other electronic devices, as parts of and/or as software applications thereon. Such electronic devices, components, and applications may use any suitable type of processor-based platform, and typically will involve a processor coupled to a computer-readable medium, such as memory, and include hardware and user interface components. The computer-readable medium can contain stored data and/or program code or applications, such as applications 114, that can be executed by the processor. The computing and electronic devices themselves may have differing characteristics. They may include cell phone devices, mobile phone devices, smart phone devices, pagers, notebook computers, personal computers, digital assistants, personal digital assistants, digital tablets, laptop computers, Internet appliances, blackberry devices, Bluetooth devices, standard telephone devices, fax machines, other suitable computing devices, or virtually any other electronic device. Additional components in the devices and applications may differ and provide various functionalities. The applications and devices may be connected to a network, such as the Internet 124, in a variety of ways. The network is not limited to any particular type of network nor is it limited to a single network. For example, the network could involve the Internet, a LAN, a WAN, a private network, a virtual network, or any combination of network types. The connection may be virtually any type including network, line, and/or wireless connections.

III. Data Harvester

A. Video Acquisition

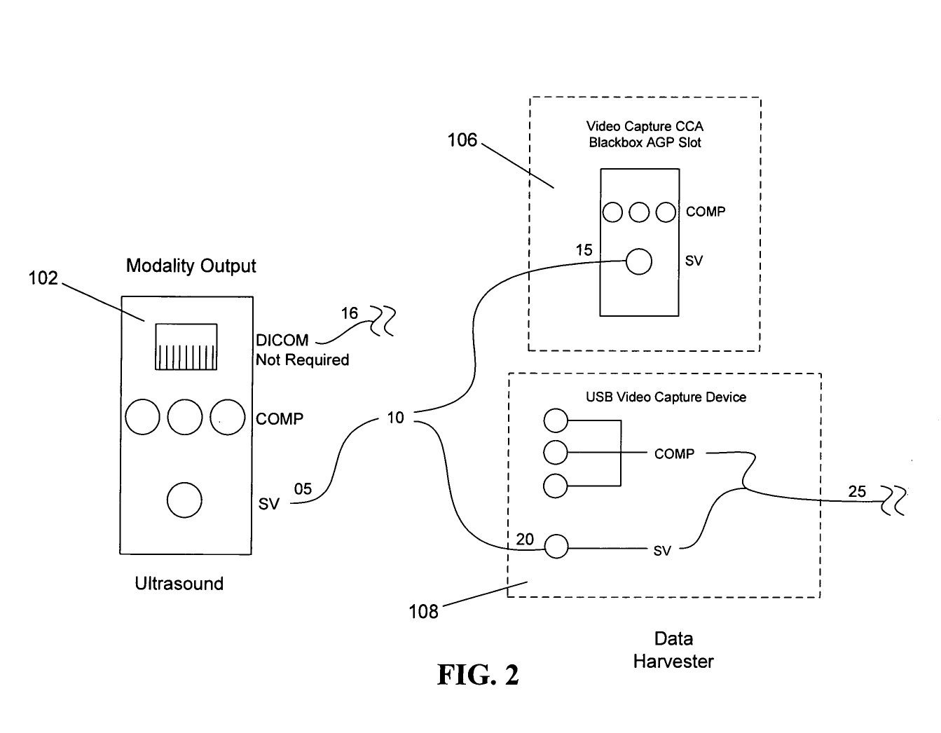

FIG. 2 illustrates the interaction between the ultrasound 102 output and data harvester 104 in one embodiment of the present invention. The technologist or radiologist, cardiologist, or other specialist is enabled to send video clips and DICOM data to the data harvester 104. The data harvester 104 captures and may encode video taken directly from the ultrasound 102 output. A video capture CCA blackbox AGP slot 106 is configured to capture and encode video clips, receive DICOM data, such as patient demographics, measurements, etc., and create an electronic preliminary report.

The data harvester 104 may be configured to receive DICOM data only in cases where certain requirements are satisfied. For example, it may only receive DICOM data if an Ethernet connection is ready, the system is DICOM enabled, there is an Application Entity (AE) title, and/or a static IP address is being used. The system may also be configured to identify and correct DICOM send issues. If the system is unable to satisfy necessary DICOM requirements, the technologist, radiologist, cardiologist, or other specialist may type or otherwise enter the relevant DICOM data into the data harvester interface and proceed with the study.

B. Video Acquisition Using a Foot Pedal

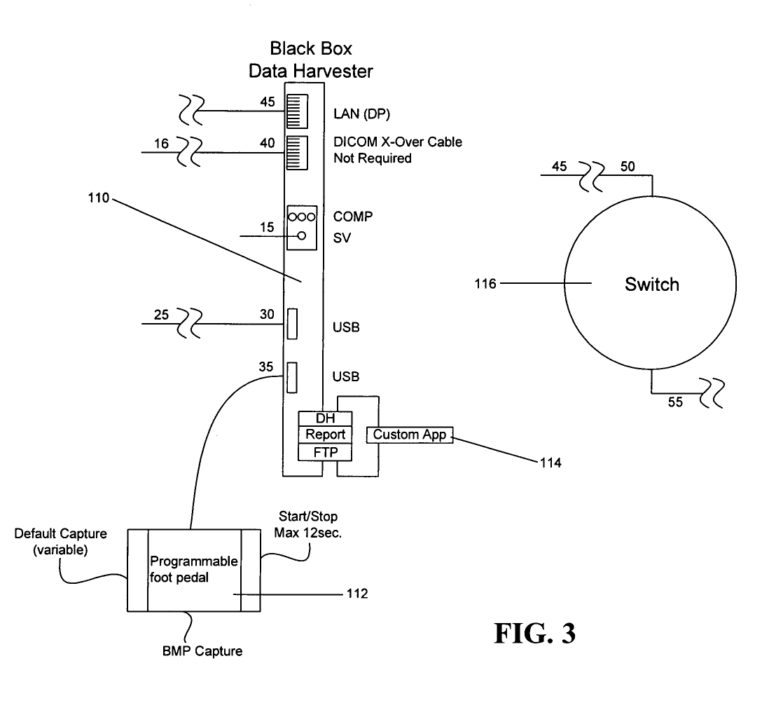

Referring now to FIGS. 1, 2 and 3, in certain embodiments the data harvester 104 includes a USB video capture device 108 that is controlled by a foot pedal 112. The video acquisition process utilizes the programmable three-button foot pedal 112 to capture images. The foot pedal 112 gives the technologist the freedom to use both hands while performing the study. According to certain embodiments, the data harvester 104 is part of a computing device and the foot pedal 112 attaches to the data harvester's 104 USB2 port. The foot pedal 112 may include one or several foot-operated buttons. According to certain embodiments, the foot pedal includes three buttons, one for default capture, one for still capture, and one for start/stop capture. Default capture or timed capture involves a predefined start and stop capture setting based on a period of time that is configured prior to the study. For example, a default time of 3 or 4 seconds may be set prior to starting the study. Still capture enables capture of a still image of the current study. Start/stop capture enables manual control of the start and stop of the video capture. In many cases, it will allow extension of a clip beyond the default capture setting. In some cases, the length of the start/stop video clip will have a maximum. For example, the start/stop could be limited to a maximum of 12 seconds.

C. Video Acquisition and Multi-Encoding

To accommodate for various Internet connection speeds experienced by users, the data harvester 104 may include software or other components to multi-encode or compress the captured video clips. The type of multi-encoding compression may be based on the user's actual Internet connection speed. This provides the best video stream based on actual system conditions.

D. Preliminary Report Preparation

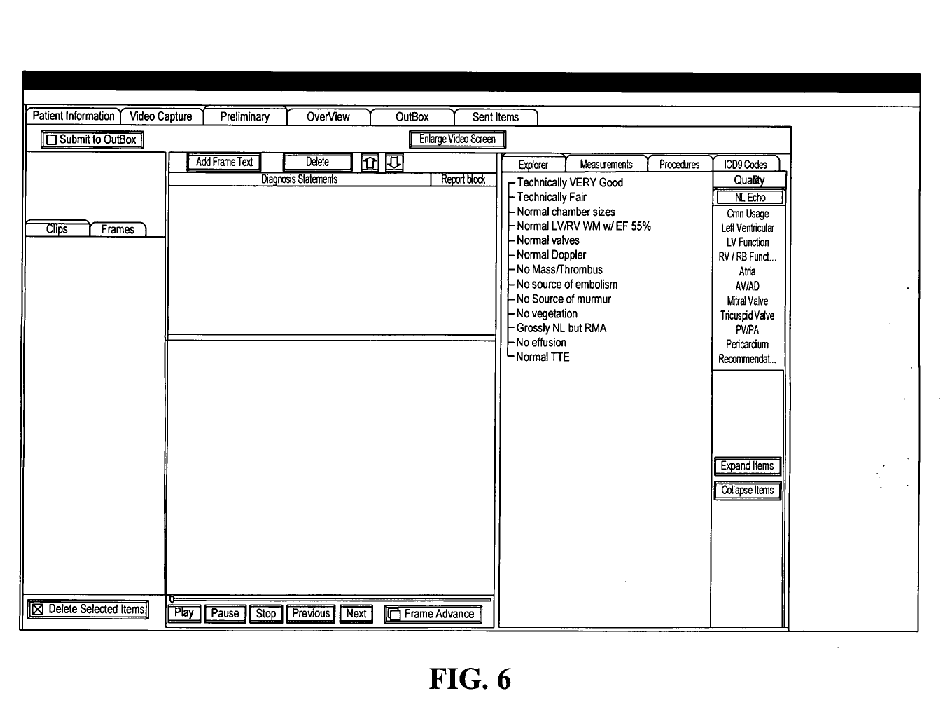

After completion of the video acquisition process on the data harvester 104, the technologist, radiologist, cardiologist, or other specialist may utilize an integrated report generator to create a comprehensive diagnosis report. The report generator may exist as a software application, such as one of applications 114 as part of the black box data harvester 110, or may exist elsewhere on the system or in other forms. To assist in generating the electronic, preliminary report, a user interface of the report generator provides convenient access to relevant information and guides the user through the necessary report creation steps. An exemplary user interface display is illustrated in FIG. 6. In certain embodiments, four primary fields make up the report generating process and assist in creating a preliminary report. These fields include: a video thumbnails field, an embedded video player field, a diagnosis statement field, and a diagnosis explorer statement tree field. The video thumbnails field allows one to select one or more appropriate video clips to be included as part of the preliminary report. The embedded video player field allows one to review video clips to be included in the preliminary report. Note that the preliminary report may include the video clips or may be sent with them. The invention is not limited to any particular image or report format.

The diagnosis statement field will typically be blank initially and is populated by the explorer statement tree selections. Of course, the technologist, radiologist, cardiologist, or other specialist may manipulate the fields after they are populated or may forgo the pre-selected statements and enter or type an entry. The diagnosis explorer statement tree allows the user to simply select a statement from a tree selection menu to initiate the report generation process. In certain embodiments, the user interface associates each statement with a check box that may be selected or not selected by the user. Double clicking and selecting a check box in the diagnosis explorer tree field will populate the diagnosis statement field with the selected statement. These features make the report generating process simpler, more efficient, and quicker. This results in quicker results and improved patient care. Selection of various check boxes may also prompt the user for additional checkboxes or otherwise provide the user the opportunity or requirement of providing additional or related details and explanations.

Routing the selected statements to a section (e.g. 2 d/M mode) or multiple sections (e.g. 2 d/M mode and Doppler) on the report is as easy as checking an action box. The action box routes the statement to a section on the report. The terms 2 d/M mode and Doppler refer to exemplary types of ultrasound image/data modes. 2D mode may provide a simple black and white picture or image of an organ such as the heart. M Mode may provide a one-dimensional moving image. In such an image of a heart, waves will typically indicate structures in the heart, such as the valves, chambers, and walls. Doppler is an ultrasound test that looks at blood flow through the heart. It will typically have color to show blood moving away and to the device used to capture the image. Other types of ultrasound image/data modes are possible.

Embodiments that are DICOM enabled send data to the appropriate diagnosis explorer field, reducing the time spent generating the report. In embodiments that are not DICOM enabled, simply selecting a preexisting statement and typing the DICOM data produces a report that is legible and easily reproducible.

E. Preliminary Report Overview

Embodiments of the present invention may also allow the technologist, radiologist, cardiologist, or other specialist to review his or her statements and correct entries on the preliminary report, such as an echocardiogram report. This overview process may result in a more polished, electronic preliminary report. A printer may be attached to or otherwise be accessible from the data harvester 104 so that one can print multiple copies of the preliminary report for any needed purpose, including hard copy reviewing, storing as medical records, sending physical copies to a referring physician for review, or sending to a patient.

F. Preliminary Report Submit Process

Referring now to FIGS. 1, 2, and 3, once the radiologist, cardiologist, or other specialist is satisfied with the output of the preliminary diagnosis report, video clips, and patient information, the study is packaged and submitted to the data harvester 104 outbox. The study is sent to the data packager 122 for transmission. The study package is a set of instructions wrapped around the study and processed by the data packager 122. This wrapper may contain routing information, database information, study data and information on the location where the study resides, among other things. In some embodiments, this wrapper is necessary for further AIM processing. In others, it is not necessary. In the cases where an operator is on the road with limited or no Internet or network access or the facility is experiencing network connectivity issues, the process may simply capture and hold studies until connectivity is established. The user may or may not be alerted.

IV. The Data Packager

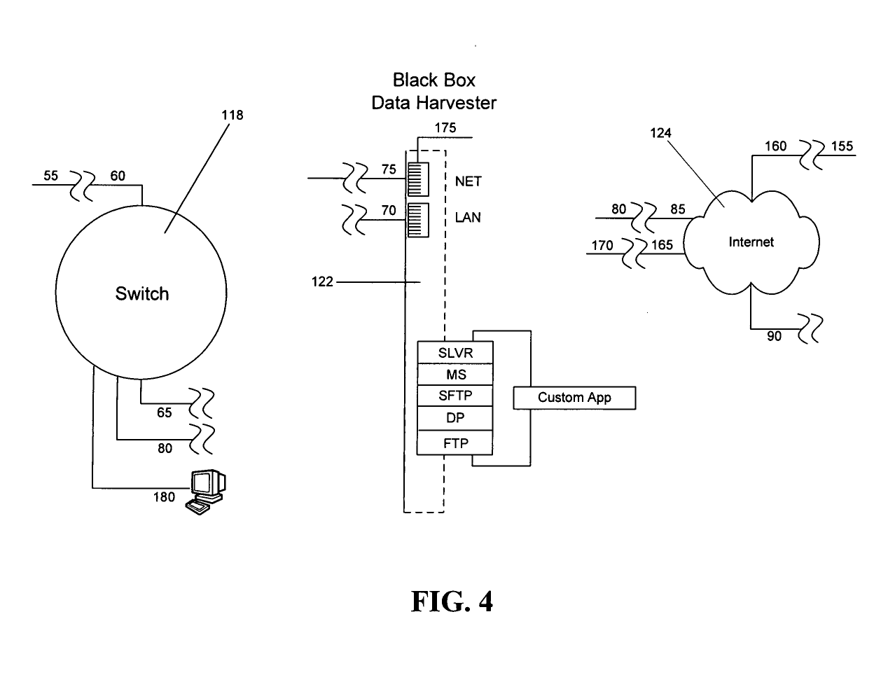

FIG. 4 illustrates a data packager 122 and a switch 118 of certain embodiments of the present invention. The data packager 122 controls communications between the data harvester 104 and one or more AIM components 132. According to certain embodiments, the data packager is designed as a multi-homed, multi-tasking server. In some embodiments, under normal local area network operations, the standard FTP protocol is the communication link between the data harvester 104 and the data packager 122. However, transmission between the data packager 122 and the AIM components 132 may involve transmission over public networks such as the Internet 124. Accordingly, in some embodiments, such transmission will use secure FTP protocol to encrypt control and payload information. Switch 118, like switch 116, adds versatility to the system, allowing for simplified modifications to the system configuration and system expansion. In some embodiments, switches may not be necessary and in others the system may be configured with switches in different physical or logical locations.

According to certain embodiments, the data packager 122 functions as a short-term image repository and streaming media server. For streaming video, the data packager 122 serves up the video stream locally on a LAN in some circumstances and remotely to AIM components in other circumstances. Serving the video clips from the data packager 122 improves video streaming for physicians on the local system. The data packager operations may also include providing an FTP location for files received by the data harvester 104, controlling a data harvester access control list, controlling data harvester maintenance and software updates, secure FTP communication with AIM components, video repository routing, establishing database connectivity with AIM components, and/or controlling enterprise and facility communications.

V. Archiving and Image Management (AIM)

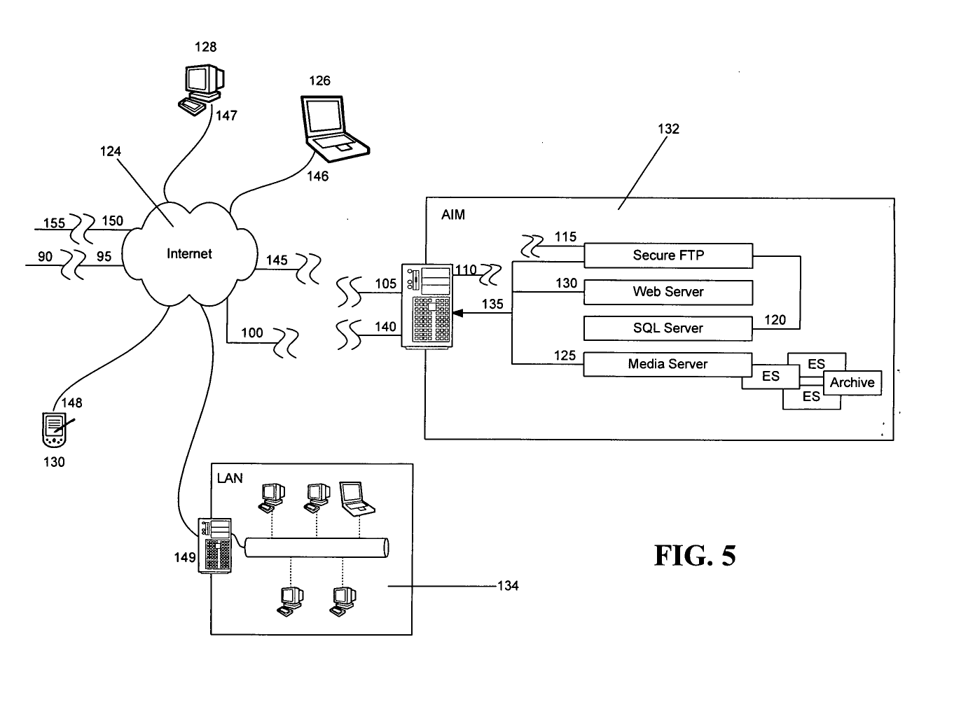

FIG. 5 illustrates an exemplary network that includes an AIM component in accordance with one embodiment of the present invention. Various embodiments of the present invention may include or relate to Archiving and Image Management (AIM) systems, components, and methods. An exemplary AIM system described herein combines different technologies to create an electronic structured report, stream videos, and provide a database and/or data warehouse. Components of the exemplary AIM system include a database server, a streaming media server, a web server, an SFTP server, and a data warehouse. These components give a reading physician the ability to access patient study data and images, such as cardiac ultrasound studies anywhere in any suitable circumstance. Generally, the information will be available to any physician anytime he or she has access to the Internet. These components give the remotely located referring physician the same access to data as the locally located reading physician.

AIM removes the geographic boundaries associated with many current processes, provides a structured workflow environment improving accuracy, increases the number of studies that may be conducted per day, and enhances patient care. Such components provide for economies of scale in that a single physician may review, analyze and generate reports of video captured by multiple technicians at multiple remote locations. Reports generated in this manner may be transmitted to the patient or patients physician according to certain embodiments of the invention. AIM may include a user interface that allows a user to capture, report and manage cardiac ultrasound studies across a wide range of networks. It also extends physicians' ability to collaborate by allowing multiple remotely located physicians access to data concurrently.

The AIM components make patient studies, reports, and other medical information available on the web and facilitates the reporting process by implementing a structured workflow. The components may utilize FTP, SFTP, SQL, WMS, WME, WMP, and the Internet, among other things, to receive and manage captured video clips and facilitate report generation and workflow. The process improves accuracy, workflow, and provides mechanisms for tracking studies.

The AIM components and systems may include an SQL or database server in which captured data is written. For example, a study may be recorded in such a database and thereafter made available to the applicable persons.

The AIM components and systems may include a Media server that works with a web server to authenticate users that have access to the studies and reports. The web server may provide a physicians' portal, providing a physician convenient access to the studies and reports to which he or she has access permission. This allows the physician to review a preliminary report and all relevant information including the stored video clips. It also allows the physician to create and upload a final report. The portal may provide a convenient user interface offering the physician predefined statements in checkbox form, similar to the preliminary report generator. Alternatively, the physician may use a separate application or a general word processor to generate the final report. The physician's final report may include an electronic or digital signature. The web server may also be used as a billing portal, a medical reports portal, and/or an administrative support portal. For example, an administrative support portal could be used for assigning user access, releasing studies, and routing studies.

To accommodate clients with limited Internet connectivity speed, patient studies can be downloaded. Downloading speeds up the retrieval and play of the large clips and in certain embodiments allows a user to bypass the media server. The downloaded studies may work in conjunction with a reading physician web portal. Through such a physician portal, the physician selects the study to read and download. The study is retrieved from the repository and pushed to a specific folder on the connected computer. The web portal may also be used to remove the downloaded study after use, through, for example, a ‘remove studies’ command.

Embodiments of the present invention may also facilitate workflow by sending electronic notifications to appropriate individuals to encourage prompt review of results and report preparation. For example, a requesting physician could receive an electronic mail message notifying him or her that a preliminary report is available on the system for his or her review. The notification message could contain a copy of or link to the relevant patient report or study.

VII. Alternative Embodiments

The structures and processes described above illustrate exemplary embodiments of inventive concepts included in the present invention. Other systems and processes are possible. While the invention has been described in detail with particular references to these particular embodiments, variations and modifications can be affected within the spirit and scope of the invention as described in this document. For example, the techniques of the present invention may also be used along with historical patient data, systems, and methods. Nothing in this specification is meant to limit, expressly or implicitly, the plain meaning of the terms used herein.

1. A video-recorded patient study reporting system, comprising:
a data harvester that receives digital video clips;
a preliminary report generator comprising (a) a user interface for creating a preliminary report of one or more diagnosis statements, and (b) a review engine that allows review and correction of the one or more statements;
a data packager that packages and transports the preliminary report and video clips; and
a data center for receiving the preliminary report and offering authorized users electronic access through one or more networks to the preliminary report.

2. The medical image reporting system of claim 1, wherein the data packager further provides electronic access to the preliminary report and video clips through one or more networks.

3. The medical image reporting system of claim 2, wherein the data packager further comprises a short-term image repository and streaming media server for providing the electronic access to the video clips.

4. The medical image reporting system of claim 1, wherein the preliminary report generator further comprises a connection to one or more medical information systems to retrieve general patient information.

5. The medical image reporting system of claim 1, further comprising a final report generator for providing a final report to an interested party.

6. The medical image reporting system of claim 1, wherein the data packager packages the preliminary report and video clips using a set of instructions related to a study.

7. The medical image reporting system of claim 6, wherein the set of instructions comprises routing information, database information, study data and information on the location where the study resides.

8. The medical image reporting system of claim 1, wherein the preliminary report is DICOM compatible.

9. A method of managing a video-recorded patient study comprising:
receiving one or more digital video clips;
generating a preliminary report comprising one or more diagnosis statements;
reviewing the diagnosis statements of the preliminary report;
submitting the preliminary report and one or more video clips to a data packager;
packaging and transporting the preliminary report and the one or more video clips from the data packager to a data center;
offering electronic access to the preliminary report to authorized user from the data center and through one or more networks; and
providing a final report to an interested party.

10. The method of claim 9, further comprising providing electronic access to the preliminary report and video clips from the data packager through one or more networks.

11. The method of claim 9, further comprising retrieving general patient information from one or more medical information systems.

12. The method of claim 9, further comprising packaging the preliminary report and video clips using a set of instructions related to the study.

13. The method of claim 12, wherein the set of instructions comprises routing information, database information, study data and information on the location where the study resides.

14. A method of managing a video-recorded ultrasound patient study comprising:
acquiring an ultrasound video;
evaluating the ultrasound video by (a) presenting the ultrasound video to a first user through a user interface; (b) accepting one or more diagnosis statements; and (c) storing the one or more diagnosis statements as a preliminary report;
reviewing the preliminary report by (a) presenting the ultrasound video to a second user through a user interface, (b) presenting the one or more diagnosis statements to the second user through the user interface, (c) allowing changes to the one or more diagnosis statements by the second user, and (d) storing a revised preliminary report;
at a data packager, packaging and encrypting the ultrasound video and revised preliminary report from a data packager to an archiving and image management (AIM) data center;
at the AIM data center, decrypting the ultrasound video and revised preliminary report, storing the ultrasound video and revised preliminary report in one or more databases, and providing the stored ultrasound video and revised preliminary report to authorized users through one or more networks.

15. The method of claim 14, further comprising replicating the ultrasound video and revised preliminary report at one or more additional AIM data centers.

16. The method of claim 14, wherein the data packager further provides local access to the ultrasound video and revised preliminary report.

17. The method of claim 14, wherein the data packager further provides access to the ultrasound video and revised preliminary report when they are not available from an AIM data center.

18. The method of claim 14, further comprising allowing one or more of the authorized users to generate a final report by (a) providing the ultrasound video and revised preliminary report through the one or more networks to one or more of the authorized users, (b) allowing changes to the one or more diagnosis statements, (c) storing the final report at the AIM data center, and (d) replicating the final report at additional AIM data centers.

19. The method of claim 14, further comprising sending electronic notifications to one or more of the authorized users when user action is requested.

20. A method of providing archiving and image management (AIM) from a data center, comprising:
storing videos and reports at one or more database servers;
providing the stored videos and reports to authorized users through one or more networks, wherein, for a user request, one or more videos and reports are provided by:
(a) authenticating the user;
(b) determining the connection speed of the user;
(c) encoding the one ore more videos and reports based on the connection speed; and
(d) providing the encoded one or more videos and reports to the user.

21. The method of claim 20, wherein providing the encoded one or more videos and reports comprises allowing the user to download the one or more videos and reports.

22. A device for use in capturing video comprising;
a first foot-operated component that controls a first form of video capture; and
a second foot-operated component that controls a second form of video capture.

23. The device of claim 22, wherein the first foot-operated component allows selection of video capture for predefined amount of time.

24. The device of claim 22, wherein the first foot-operated component allows selection of still image capture.

25. The device of claim 22, wherein the first foot-operated component allows selection of the start and stop of the video capture.
